SEQUENCE LISTING (32 sequences)

SEQ ID NO: 1 - DNA, 19 bp, Homo sapiens
gttggagctgatggcgtag

SEQ ID NO: 2 - DNA, 19 bp, Homo sapiens
gttggagctgctggcgtag

SEQ ID NO: 3 - DNA, 19 bp, Homo sapiens
gttggagctgttggcgtag

SEQ ID NO: 4 - DNA, 19 bp, Homo sapiens
gttggagctagtggcgtag

SEQ ID NO: 5 - DNA, 19 bp, Homo sapiens
gttggagctcgtggcgtag

SEQ ID NO: 6 - DNA, 19 bp, Homo sapiens
gttggagcttgtggcgtag

SEQ ID NO: 7 - DNA, 19 bp, Homo sapiens
gttggagctggtgacgtag

SEQ ID NO: 8 - DNA, 19 bp, Homo sapiens
gttggagctggtggcgtag

SEQ ID NO: 9 - DNA, 19 bp, Artificial Sequence
  Description of Artificial Sequence Synthetic PNA probe
ctacgccactagctccaac

SEQ ID NO: 10 - DNA, 13 bp, Artificial Sequence
  Description of Artificial Sequence Synthetic PNA probe
acgccatcagctc

SEQ ID NO: 11 - DNA, 13 bp, Artificial Sequence
  Description of Artificial Sequence Synthetic PNA probe
acgccagcagctc

SEQ ID NO: 12 - DNA, 13 bp, Artificial Sequence
  Description of Artificial Sequence Synthetic PNA probe
acgccaacagctc

SEQ ID NO: 13 - DNA, 13 bp, Artificial Sequence
  Description of Artificial Sequence Synthetic PNA probe
acgccacgagctc

SEQ ID NO: 14 - DNA, 13 bp, Artificial Sequence
  Description of Artificial Sequence Synthetic PNA probe
acgccacaagctc

SEQ ID NO: 15 - DNA, 13 bp, Artificial Sequence
  Description of Artificial Sequence Synthetic PNA probe
cctacgtcaccag

SEQ ID NO: 16 - DNA, 13 bp, Artificial Sequence
  Description of Artificial Sequence Synthetic PNA probe
acgccaccagctc

SEQ ID NO: 17 - DNA, 19 bp, Artificial Sequence
  Description of Artificial Sequence Synthetic PNA probe
ctacgccatcagctccaac

SEQ ID NO: 18 - DNA, 19 bp, Artificial Sequence
  Description of Artificial Sequence Synthetic PNA probe
ctacgccagcagctccaac

SEQ ID NO: 19 - DNA, 19 bp, Artificial Sequence
  Description of Artificial Sequence Synthetic PNA probe
ctacgccaacagctccaac

SEQ ID NO: 20 - DNA, 19 bp, Artificial Sequence
  Description of Artificial Sequence Synthetic PNA probe
ctacgccacgagctccaac

SEQ ID NO: 21 - DNA, 19 bp, Artificial Sequence
  Description of Artificial Sequence Synthetic PNA probe
ctacgccacaagctccaac

SEQ ID NO: 22 - DNA, 19 bp, Artificial Sequence
  Description of Artificial Sequence Synthetic PNA probe
ctacgtcaccagctccaac

SEQ ID NO: 23 - DNA, 13 bp, Artificial Sequence
  Description of Artificial Sequence Synthetic PNA probe
acgccactagctc

SEQ ID NO: 24 - DNA, 19 bp, Artificial Sequence
  Description of Artificial Sequence Synthetic PNA probe
ctacgdcaddagctccaac

SEQ ID NO: 25 - DNA, 16 bp, Artificial Sequence
  Description of Artificial Sequence Synthetic PNA probe
gatttctctgtagcta

SEQ ID NO: 26 - DNA, 13 bp, Artificial Sequence
  Description of Artificial Sequence Synthetic PNA probe
gatttcactgtag

SEQ ID NO: 27 - DNA, 16 bp, Homo sapiens
tagctacagagaaatc

SEQ ID NO: 28 - DNA, 16 bp, Homo sapiens
tagctacagtgaaatc

SEQ ID NO: 29 - DNA, 27 bp, Artificial Sequence
  Description of Artificial Sequence Synthetic primer
cctcacagtaaaaataggtgattttgg

SEQ ID NO: 30 - DNA, 26 bp, Artificial Sequence
  Description of Artificial Sequence Synthetic primer
cacaaaatggatccagacaactgttc

SEQ ID NO: 31 - DNA, 24 bp, Artificial Sequence
  Description of Artificial Sequence Synthetic primer
gcctgctgaaaatgactgaatata

SEQ ID NO: 32 - DNA, 23 bp, Artificial Sequence
  Description of Artificial Sequence Synthetic primer
ttagctgtatcgtcaaggcactc

(19) United States
(12) Patent Application Publication
(10) Pub. No.: US 2016/0265064 A1
(43) Pub. Date: Sep. 15, 2016

(54) ELECTROCHEMICAL CLAMP ASSAY

(71) Applicant: The Governing Council of the University of Toronto, Toronto (CA)

(72) Inventors: Jagotamoy Das, Scarborough (CA); Ivaylo Ivanov, Toronto (CA); Edward Hartley Sargent, Toronto (CA); Shana O. Kelley, Toronto (CA)

(21) Appl. No.: 15/067,392

(22) Filed: Mar. 11, 2016

Related U.S. Application Data
(60) Provisional application No. 62/131,434, filed on Mar. 11, 2015.

(51) Int. Cl. C12Q 1/68; G01N 27/327; G01N 27/416

(52) U.S. Cl. CPC C12Q 1/6886; G01N 27/4166; G01N 27/3278; C12Q 2600/156; C12Q 2600/158

(57) ABSTRACT

Described herein are systems and methods for electrochemically detecting a variant of a target sequence in a sample, the target sequence being present as a plurality of variants within the sample, the system comprising an electrode comprising a first probe on its surface, said probe being capable of binding a first variant of the target sequence, and a second probe capable of binding a second variant of the target sequence, wherein the second probe is added to the sample, thereby preventing binding of the second variant to the first probe. Also disclosed herein are kits for electrochemical detection of target sequences.

### CROSS-REFERENCE TO RELATED APPLICATIONS

This application claims the benefit of U.S. Provisional Application No. 62/131,434, filed on Mar. 11, 2015, which is hereby incorporated herein by reference in its entirety.

### BACKGROUND

Cell-free nucleic acids (cfNAs) released from tumors are present in the blood of patients with cancer as they have the potential to act as markers for cancer diagnosis and management. Cancer patients often have higher levels of cfNAs than do healthy individuals. Examples of tumor-related mutated sequences, include the Kirsten rat sarcoma-2 virus (KRAS) gene that is associated with lung cancer, colorectal cancer, and ovarian cancer, and the BRAF gene associated with melanoma. The ability to detect mutated sequences (e.g., KRAS and BRAF) linked to cancer could allow specific monitoring of tumor-related sequences. Furthermore, detection of cfNAs in plasma or serum could serve as a liquid biopsy, replacing tumor tissue biopsies in certain diagnostic applications.

The existing approaches that are able to monitor cfNAs rely on the polymeric chain reaction (PCR) or DNA sequencing. The implementation of DNA sequencing is usually too expensive for routine clinical use, and the slow turnaround time (2-3 weeks) is not ideal for optimal treatment outcomes. PCR is not typically effective for the detection of point mutations, but the introduction of peptide nucleic acid (PNA) clamps boosts the accuracy of this approach. The PNAs serve as sequence-selective clamps that prevent amplification of wild-type DNA during clamp PCR, and the mutated sequence is then selectively amplified. PCR is prone to interference from the components of biological samples, and as such clamp PCR is not able to detect cfNA mutations in blood or serum samples directly. Instead the samples require pre-processing from large-volume samples (e.g. >5 ml) and then purification of the cfNAs. Additionally, clamp PCR can introduce bias based on the amplification efficiency of different sequences.

Chip-based methods leveraging electronic or electrochemical readout represent attractive alternatives for clinical sample analysis because they are amenable to automation, are low cost and have the potential for high levels of multiplexing and sensitivity. This type of testing approach has been applied successfully to the analysis of cancer as well as a variety of infectious pathogens, but the feasibility of analyzing cfNAs for cancer-related mutations in clinical samples has not been established. Previous efforts to achieve point mutation detection based on electrochemical methods have relied on stringent control of assay conditions or mismatch-sensitive enzymes, but these types of approaches do not yield significant selectivity in heterogeneous patient samples where a mutated sequence may be outnumbered by a high level of the wild-type sequence A method that is more sensitive, specific and efficient than the existing methods, and one which is also able to detect cfNA mutations directly in serum or blood, is therefore desirable.

### SUMMARY

Disclosed herein are systems, devices, and methods for the electrochemical detection of a first variant of a target sequence in a sample. Electrochemical sensors are functionalized with molecules that render them specific for a nucleic acids sequence of interest, and a series of probes (“clamp”) molecules are used to eliminate cross-reactivity with wild-type DNA and with deliberately-selected mutants. The electrochemical clamp assay is an advantageous technique over PCT as it can successfully be applied to unpurified serum samples. This is of particular benefit because it can reduce bias in the pool of sequences that are isolated. In addition, very small volumes (e.g., less than about 5 mls, less than about 4 mls, less than about 3 mls, less than about 2 mls, less than about 1 ml, less than about 500 μl and less than about 50 μL) of serum can be analyzed with this approach, whereas 5 ml of serum is required to yield enough purified cfNA for analysis via clamp PCR. Moreover, the analysis time can be shorter (e.g., about 5 minutes, less than about 5 minutes, about 4 minutes, about 3 minutes, about 2 minutes or about 1 minute) than the 2-3 hours required by PCR, and the several days required by DNA sequencing techniques. The electrochemical clamp assay is therefore suitable for a point-of-care device.

According to one aspect, there is provided a detection system for electrochemically detecting a variant of a target sequence in a sample, the target sequence being present as a plurality of variants within the sample, the system comprising: an electrode comprising a first probe on its surface, said first probe being capable of binding a first variant of the target sequence and; a second probe capable of binding a second variant of the target sequence, wherein the second probe is added to the sample, thereby preventing binding of the second variant to the first probe.

According to another aspect, there is provided a method for electrochemical detection of a variant of a target sequence in a sample, the target sequence being present as a plurality of variants within the sample, the method comprising: contacting an electrode comprising a first probe (e.g., an electrochemical probe) on its surface with the sample, said first probe, preferably deployed on the surface of the electrode and being capable of binding a first variant of the target sequence, adding a second probe to the sample, said second probe being capable of binding a second variant of the target sequence, thereby preventing binding of the second variant to the first probe; and measuring an electrochemical signal generated by the binding of the first variant of the target sequence to the first probe, wherein the electrochemical signal is indicative of the presence of the first variant within the sample.

According to a further aspect there is provided a point-of care diagnostic device configured to perform the method described herein.

According to another aspect there is provided a kit comprising: a biosensor comprising an electrode; a first probe affixed to surface of the electrode, said first probe being capable of binding a first variant of a target sequence in a sample, said sample containing a plurality of variants of the target sequence; a second probe, capable of binding a second variant of a target sequence in a sample containing a plurality of variants of the target sequence, thereby preventing binding of the second variant to the first probe.

According to a still further aspect there is provided a method of detecting a variant of a cancer-related sequence mutation in sample from a patient, the method comprising: contacting an electrode comprising a first probe on its surface with the sample, said first probe being capable of binding a first variant of the cancer-related sequence mutation, adding at least a second probe to the sample, said second probe being capable of binding a second variant of the cancer-related sequence mutation, thereby preventing binding of the second variant to the first probe; and measuring an electrochemical signal generated by the binding of the first variant of the cancer-related sequence mutation to the first probe, wherein the electrochemical signal is indicative of the presence of the first variant within the sample.

In some embodiments of the electrochemical detection systems and methods provided herein, the electrode is a microelectrode. In other embodiments, the microelectrode is a nanostructured microelectrode (“NME”). NMEs are microelectrodes that feature nanostructured surfaces. Surface nanotexturing or nanostructures provide the electrode with an increased surface area, allowing greater sensitivity, particularly in biosensing applications. Manufacturing of NMEs can be performed by electrodeposition. By varying the parameters such as deposition time, deposition potential, supporting electrolyte types and metal ion sources, NMEs of a variety of sizes, morphologies and compositions may be generated. In certain instances, NMEs have a dendritic or fractal structure. Exemplary NMEs for use in the systems and methods described herein are described in International Patent Publication WO2010/025547, which is hereby incorporated by reference in its entirety. In additional embodiments, the electrode is on a microfabricated chip.

Other electrode structures can also be used in the detection systems and methods described herein, including planar surfaces, wires, tubes, cones and particles. Commercially available macro- and micro-electrodes are also suitable for the embodiments described herein.

In some embodiments of the electrochemical detection systems and methods provided herein the first and the second probe is an oligonucleotide. In some embodiments the oligonucleotide is a peptide nucleic acid (PNA).

In some embodiments of the systems and methods provided herein the target sequence to be detected is a cell-free nucleic acid (cfNA). Most DNA and RNA in the body are located within cells, but a small amount can be found circulating freely in the blood. A substantial proportion of such DNA and RNA molecules (cfNAs) are thought to come from cells undergoing apoptosis or necrosis, which release their contents into the blood stream. The analysis of cfNAs offers a non-invasive approach for the diagnosis of a variety of diseases/disorders that are capable of being diagnosed using genetic analysis. In some embodiments of the systems and methods described herein, cfNAs (present at significant levels in the blood of cancer patients) are analyzed to reveal the mutational spectrum of a tumor without the need for invasive sampling of tissue. Non-limiting examples of cfNAs released from tumors that can be detected using the systems and methods as herein described include the Kirsten rat sarcoma-2 virus (KRAS) gene that is associated with lung cancer, colorectal cancer, and ovarian cancer, and the BRAF gene associated with melanoma. In other embodiments of the systems and methods described herein, analysis of fetal-derived cfNAs found within the maternal blood is useful for detecting and monitoring fetal diseases and pregnancy-associated complications. In some other embodiments of the systems and methods described herein, the quantitation of elevated levels of cfNAs provides an indication of the level of clinical severity of acute medical emergencies, including trauma and stroke.

The patient sample can be a blood sample, for example a whole blood sample, a plasma sample or a serum sample. In specific embodiments the electrochemical detection system enables direct analysis of cancer-related mutations in cfNAs from unprocessed patient serum samples.

### BRIEF DESCRIPTION OF THE DRAWINGS

The foregoing and other objects and advantages will be apparent upon consideration of the following detailed description, taken in conjunction with the accompanying drawings, in which like reference characters refer to like parts throughout, and in which:

FIG. 1 depicts a schematic of the electrochemical detection of a target;

FIG. 2 depicts an electrochemical readout indicating the presence/absence of a target;

FIG. 3 depicts the specificity of the electrochemical detection according to a first implementation;

FIG. 4 depicts the specificity of the electrochemical detection according to a second implementation;

FIG. 5 depicts detect limits using clamp PCR.

FIG. 6 depicts analysis of cell-free nucleic acids (cfNAs) to identify whether the electrochemical detection is of genomic DNA or transcribed RNA.

### DETAILED DESCRIPTION

To provide an overall understanding of the systems, devices, and methods described herein, certain illustrative implementations will be described.

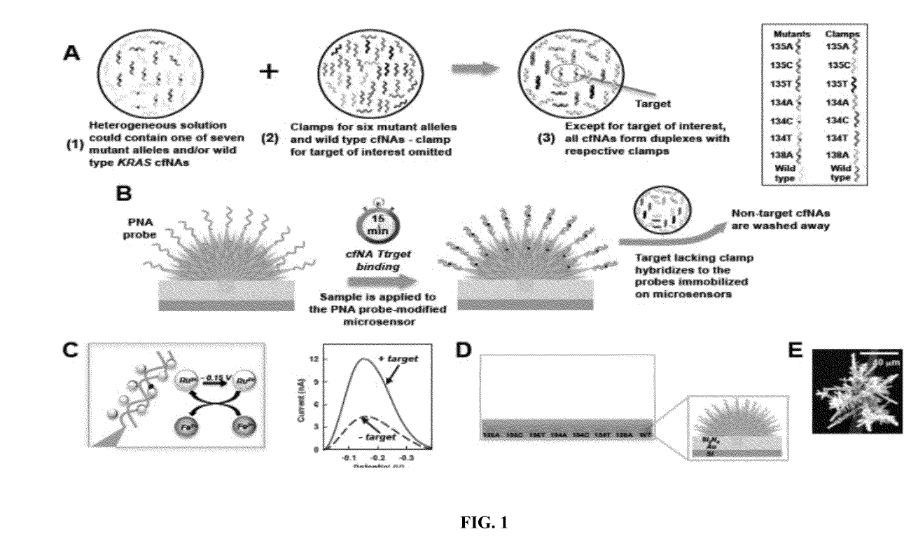

FIGS. 1-5 illustrate non-limiting examples of systems and methods for electrochemically detecting a variant sequence amongst a plurality of variant sequences within a biological sample.

FIG. 1 depicts a schematic of the use of an electrochemical clamp assay for specific detection of a cfNA mutation of the Kirsten rat sarcoma-2 virus (KRAS) gene, referred to as the 134A mutant. The KRAS gene has 7 mutations at codons 12 and 13 of 2 exons, which are denoted 135A, 135C, 135T, 134A, 134C, 134T, and 138A, as shown below:

135A allele
DNA GTT GGA GCT GAT GGC GTA G
135C allele
DNA GTT GGA GCT GCT GGC GTA G
135T allele
DNA GTT GGA GCT GTT GGC GTA G
134A allele
DNA GTT GGA GCT AGT GGC GTA G
134C allele
DNA GTT GGA GCT CGT GGC GTA G
134T allele
DNA GTT GGA GCT TGT GGC GTA G
138A allele
DNA GTT GGA GCT GGT GAC GTA G
wild-type (135G) allele
DNA GTT GGA GCT GGT GGC GTA G

A given patient sample may contain one of the 7 mutant alleles and a large amount of wild-type nucleic acids (NAs), as illustrated in FIG. 1A. Mutated KRAS alleles are associated with lung cancer, colorectal cancer, and ovarian cancer, and the efficacies of several therapies are affected by mutations in this gene. It is therefore of therapeutic benefit to be able to qualitatively and quantitatively detect the presence and/or absence of a specific mutation of the KRAS alleles.

Using photolithographic patterning, an array of forty sensors is defined to form a bioelectronic integrated circuit (IC) (FIG. 1). A SiO₂-coated silicon wafer is provided with contact pads and electrical leads, and a layer of Si₃N₄ is then deposited to passivate the top surface of the chip. In order to provide a template for the growth of electrodeposited sensors, photolithography is used to form 5 pm apertures in the top passivation layer. Au electrodeposition at locations determined by the opened apertures is used to grow three-dimensional microstructures for subsequent biosensing. The microstructured sensors protrude from the surface and reach into solution, with their size and morphology programmed by deposition time, applied potential, Au concentration, supporting electrolyte, and overcoating protocol. Since nanostructures increase the sensitivity of the assay, the Au microstructures were coated with a thin layer of Pd to form finely nanostructured microelectrodes (NMEs) (FIG. 1E). Exemplary NMEs for use in the systems and methods described herein are described in International Patent Publication WO2010/025547, which is hereby incorporated by reference in its entirety. The micron-size scale of the three-dimensional electrodes increases the cross-section for interaction with analyte molecules, while the nanostructuring maximizes sensitivity by enhancing hybridization efficiency between tethered probe and the analyte in solution.

As shown in FIG. 1B, a patient sample that includes the 134A mutation is brought into contact with an IC having a nanostructured microelectrode that includes an immobilized polynucleic acid (PNA) probe (Cys-Gly-CTA CGC CAC TAG CTC CAA C) specific for the 134A mutant KRAS allele. In order to prevent the binding of any other mutant KRAS alleles (e.g., 135A, 135C, 135T, 134C, 134T, and 138A) and the wild-type allele to the probe specific for the 134A allele, a cocktail of PNA probes (“clamps”), as listed below, are added to the patient sample (FIG. 1A):

| | | N_ C terminus |
|---|---|---|
| Clamp for 135A allele | PNA | ACG CCA TCA GCT C |
| Clamp for 135C allele | PNA | ACG CCA GCA GCT C |
| Clamp for 135T allele | PNA | ACG CCA ACA GCT C |
| Clamp for 134C allele | PNA | ACG CCA CGA GCT C |
| Clamp for 134T allele | PNA | ACG CCA CAA GCT C |
| Clamp for 138A allele | PNA | CCT ACG TCA CCA G |
| Clamp for wild-type | PNA | ACG CCA CCA GCT C |

The clamps hybridize to the six non-target mutants and the wild-type sequence, sequestering them in the sample, and leaving only the 134A mutation unhybridized. Only the mutant 134A can hybridize to the immobilized probe; all other mutant alleles and the wild-type allele are blocked by their clamps and simply remain in solution and are washed away.

As shown in FIG. 1C, specific binding of the 134A mutation to the functionalized probe is detected electrochemically, with the sensor being interrogated using an electrocatalytic reporter system, for example an electrocatalytic reporter pair comprised of Ru(NH₃)₆³⁺ and Fe(CN)₆³⁻ to read out the presence of specific the 134A mutation. Ru(NH₃)₆³⁺ is electrostatically attracted to the negatively-charged phosphate backbone of nucleic acids that bind to the probes immobilized on the surface of electrodes and is reduced to Ru(NH₃)₆² when the electrode is biased at the reduction potential. The Fe(CN)₆³⁻ present in solution chemically oxidizes Ru(NH₃)₆²⁺ back to Ru(NH₃)₆³⁺ allowing for multiple turnovers of Ru(NH₃)₆³⁺, which generates an high electrocatalytic current. The difference between pre-hybridization and post-hybridization currents is used as a metric to determine target binding (typical differential pulse voltammograms (DPVs) before and after 100 fg/μL target mutant cfNA (134A) binding).

### I. Specific Point Mutation Detection

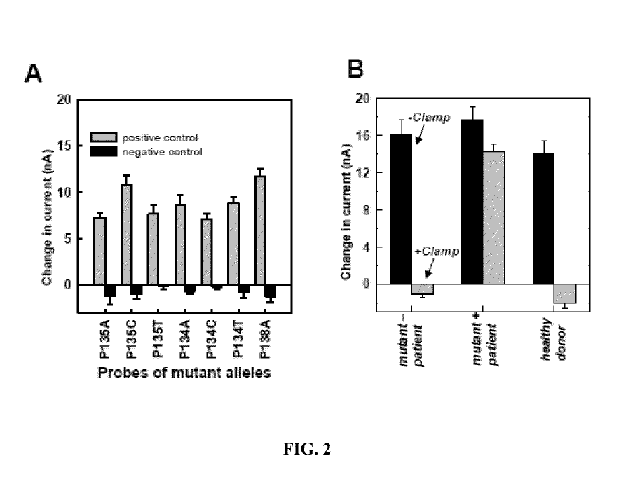

FIG. 2 illustrates the used of an IC chip to genotype seven distinct point mutation alleles of the KRAS gene that are associated with lung cancer.

The sequences of the seven mutant KRAS alleles, and of the oligonucleotides used as probes and clamps, are shown below. DNA sequences are shown 5′ to 3′, and PNA sequences are shown N to C terminus. Underlined portions denote point mutations.

Target for 135A allele
DNA GTT GGA GCT GAT GGC GTA G
Target for 135C allele
DNA GTT GGA GCT GCT GGC GTA G
Target for 135T allele
DNA GTT GGA GCT GTT GGC GTA G
Target for 134A allele
DNA GTT GGA GCT AGT GGC GTA G
Target for 134C allele
DNA GTT GGA GCT CGT GGC GTA G
Target for 134T allele
DNA GTT GGA GCT TGT GGC GTA G
Target for 138A allele
DNA GTT GGA GCT GGT GAC GTA G
Target for wild-type (135G)
DNA GTT GGA GCT GGT GGC GTA G
Probe for 135A allele
PNA Cys-Gly-CTA CGC CAT CAG CTC CAA C
Probe for 135C allele
PNA Cys-Gly-CTA CGC CAG CAG CTC CAA C
Probe for 135T allele
PNA Cys-Gly-CTA CGC CAA CAG CTC CAA C
Probe for 134A allele
PNA Cys-Gly-CTA CGC CAC TAG CTC CAA C
Probe for 134C allele
PNA Cys-Gly-CTA CGC CAC GAG CTC CAA C
Probe for 134T allele
PNA Cys-Gly-CTA CGC CAC AAG CTC CAA C
Probe for 138A allele
PNA Cys-Gly-CTA CGT CAC CAG CTC CAA C
Clamp for 135A allele
PNA ACG CCA TCA GCT C
Clamp for 135C allele
PNA ACG CCA GCA GCT C
Clamp for 135T allele
PNA ACG CCA ACA GCT C
Clamp for 134A allele
PNA ACG CCA CTA GCT C
Clamp for 134C allele
PNA ACG CCA CGA GCT C
Clamp for 134T allele
PNA ACG CCA CAA GCT C
Clamp for 138A allele
PNA CCT ACG TCA CCA G
Clamp for wild-type
PNA ACG CCA CCA GCT C

A sample including complementary mutant target, non-complementary mutants, wild-type sequence, total human RNA, and a clamp cocktail was used to measure the positive signal at electrochemical sensors functionalized with probes (P135 A, P135 C, P135 T, P134 A, P134 C, P134 T, and P138 A) corresponding to each of the mutant alleles. Sensors were challenged with mixtures of nucleic acids with (positive control) and without (negative control) mutant target of interest. The positive control contained all of the seven mutant oligonucleotides with 1 nM concentration of each, 100 nM of wild-type (WT) synthetic oligonucleotides, 50 pg/μL cfNAs from healthy donors, and seven clamps except one that is complementary for target of interest. The negative control contained all of the above except target of interest and its clamp. As shown in FIG. 2A, the negative controls did not produce a positive signal change in any of the sensors tested; in contrast, the positive samples produced current changes ranging from 7 to 12 nA. These results clearly demonstrate that the electrochemical clamp assay can specifically detect each of mutant alleles of KRAS genes.

In order to investigate whether the clamps were necessary for accurate point mutation detection, a sensor was challenged with purified nucleic acids from a wild-type patient sample, a mutant-positive patient sample, and a healthy donor in presence and absence of the clamp for the wild-type sequence. Although hybridization and washing were performed at an elevated temperature, a signal increase for all three samples was observed if the clamp for the wild-type sequence was not present in solution (FIG. 2B). In the presence of the clamp for the wild-type sequence, a positive signal change for mutant-negative and healthy donor samples was not observed, but a significant signal change was observed for the mutant-positive sample in the presence of the clamp. The change of current in the presence of clamp is slightly lower than in the absence of clamp because clamp minimizes interference from wild-type nucleic acids. These results demonstrate that the use of a clamp provides a method for the sensitive and specific detection of mutations within cfNAs.

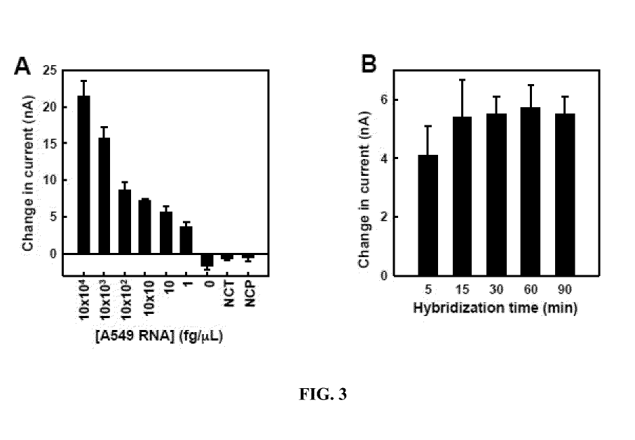

To evaluate the sensitivity of the electrochemical clamp assay, the dependence of the electrochemical signal on RNA concentration when an electrode on which a 134A mutant probe is hybridized was challenged with exosomal RNA isolated from cells derived from a lung cancer cell line (A549 cells) carrying the 134A mutation, was investigated. Concentrations of RNA ranged from 1 fg/μL to 100 pg/μL. Seven clamps except the clamp for the 134A mutation were added to the sample. Controls included (i) a blank, (ii) RNA containing the wild-type sequence isolated from cells derived from a glioblastoma cell line (exosomal RNA from U733v3 cells) (NCT in FIG. 3A), and (iii) a noncomplementary probe (NCP in FIG. 3A). (FIG. 3A). The signal change increased with increasing concentration of the target over six orders of magnitude. The assay is able to detect 1 fg/ml of A549 exosomal RNA. To evaluate the detection speed of the clamp assay, the time-dependent signal change by varying hybridization time of 10 fg/μL target RNA was investigated (FIG. 3B). The results demonstrate that the electrochemical clamp assay is capable of delivering results very rapidly, with statistically-significant signals being obtained within five minutes.

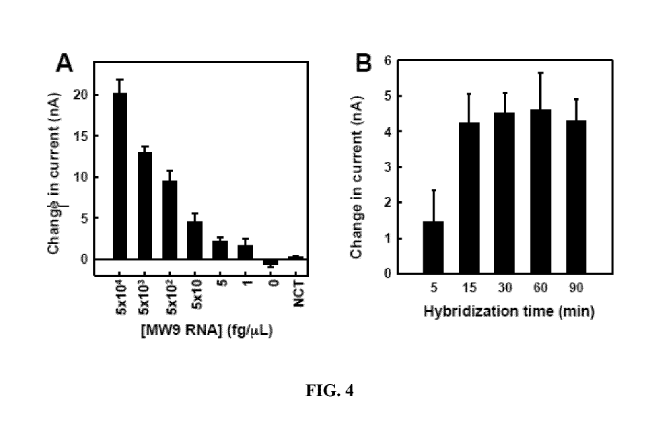

The use of the electrochemical detection system to detect other mutations in the sequence of other genes, is illustrated in FIG. 4. The specificity and sensitivity of a set of BRAF-specific probes for detecting mutations in RNA from the MW9 cell line is shown in FIGS. 4A and 4B. The sensitivity, specificity, and speed for the detection of the various BRAF mutations was similar to that demonstrated for the KRAS mutations.

### II. Patient Sample Analysis

The electrochemical clamp assay was used to analyze cfNA in processed and non-processes serum samples from lung cancer patients (KRAS) and melanoma cancer patients (BRAF) (Table 1 and 2).

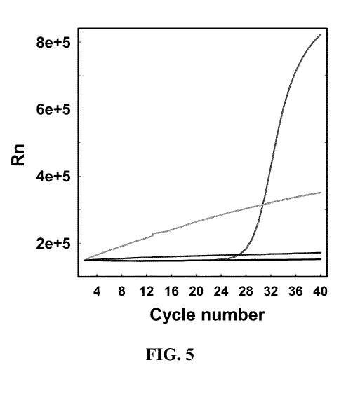

Table 1 shows the results of the analysis of KRAS mutations in cfNAs isolated and purified from lung cancer patients, and also in unprocessed lung cancer patient serum. As a control, serum from a healthy donor (HD) was processed and analyzed in the same way. A universal probe mixture (Cys-Gly-CTA CGX CAX XAG CTC CAA C (where, X=mixture of A, T, and G with unimolar ratio), allowed all possible known KRAS mutant sequences of interest to be screened in cfNAs in a single experiment. For each sample analyzed using the electrochemical clamp assay, a mean signal of −1.0±0.3 nA (plus three standard deviations) measured in the healthy donor's sample was used as a cutoff value for determining the presence or absence of the KRAS mutation. A sample with a current level higher than the cutoff value is positive for the KRAS mutation, whereas a sample with a current level lower than the cutoff value is negative for the KRAS mutation. A previously-validated clamp PCR method was used to confirm the presence or absence of the KRAS mutation. In this clamp PCR method, when ΔCt-1≧2, the sample is positive for the KRAS mutation, and when ΔCt-1<0, the sample is negative for the KRAS mutation. When 0<ΔCt-1<2, another parameter (ΔCt-2) is taken into consideration. When ΔCt-2>6, the sample is negative for the KRAS mutation. The results of the electrochemical clamp assay and clamp PCR are comparable. The electrochemical clamp assay was also used to detect KRAS mutations in unprocessed lung cancer patient serum. As demonstrated in all three assays, three (3) of the fourteen (14) lung cancer patient samples were positive for KRAS mutation. The signal changes observed in electrochemical assay for the undiluted serum is lower than in the processed samples, which is expected due to much lower levels in the purified sample. The electrochemical clamp assay is able to detect mutated KRAS in unprocessed serum, in comparison to clamp PCR method. The inability of clamp PCR to produce detectable amplification in patient sample is demonstrated in FIG. 5, which illustrates the rise in fluorescence observed as a function of PCR cycle number. Positive results were obtained when purified cell-free nucleic acids were amplified (a), but the use of undiluted serum (b) did not produce data with a clear exponential rise in signal. Diluted serum (c) and diluted and heated serum (d) were also used in an attempt to apply literature protocols to the use of PCR for this application, but negative results were also obtained.

TABLE 1
Analysis of KRAS mutations in samples from lung cancer patients

| | Purified nucleic acids | | | | | Unprocessed Serum | |
|---|---|---|---|---|---|---|---|
| | Electrochemical | | | | | Electrochemical | |
| | clamp chip*¹ | | Clamp PCR | | | clamp chip*² | |
| Samples | Δi (nA) ± sd | Assessment | ΔCt-1 | ΔCt-2 | Assessment | Δi(nA) ± sd | Assessment |
| 1 | −2.3 ± 1.0 | Wild-type | 1 | 16 | Wild-type | 0 ± 0.1 | Wild-type |
| 2 | −1.5 ± 0.5 | Wild-type | −1 | 18 | Wild-type | −0.1 ± 0.1 | Wild-type |
| 3 | 8.3 ± 1.3 | KRAS | 6 | 8 | KRAS | 2.1 ± 0.9 | KRAS |
| | | mutated | | | mutated | | mutated |
| 4 | −0.6 ± 0.7 | Wild-type | 0 | 16 | Wild-type | −0.1 ± 0.1 | Wild-type |
| 5 | −1.9 ± 0.5 | Wild-type | −2 | 16 | Wild-type | −0.4 ± 0.2 | Wild-type |
| 6 | −2.5 ± 0.9 | Wild-type | 1 | 14 | Wild-type | −0.1 ± 0.1 | Wild-type |
| 7 | −1.3 ± 0.6 | Wild-type | −4 | 14 | Wild-type | −0.4 ± 0.1 | Wild-type |
| 8 | −1.2 ± 0.5 | Wild-type | −4 | 18 | Wild-type | −0.8 ± 0.2 | Wild-type |
| 9 | 4.8 ± 0.7 | KRAS | 4 | 6 | KRAS | 1.3 ± 0.2 | KRAS |
| | | mutated | | | mutated | | mutated |
| 10 | −1.5 ± 0.2 | Wild-type | −3 | 18 | Wild-type | −0.8 ± 0.2 | Wild-type |
| 11 | −0.6 ± 0.4 | Wild-type | — | — | Wild-type | −0.1 ± 0.2 | Wild-type |
| 12 | 4.5 ± 1.4 | KRAS | 3 | 16 | KRAS | 1.4 ± 0.3 | KRAS |
| | | mutated | | | mutated | | mutated |
| 13 | −2.2 ± 0.5 | Wild-type | −2 | 16 | Wild-type | −1.2 ± 0.1 | Wild-type |
| 14 | −1.5 ± 0.3 | Wild-type | −2 | 15 | Wild-type | 0.4 ± 0.1 | Wild-type |
| HD | −1.0 ± 0.3 | Wild-type | 0 | 16 | Wild-type | −0.5 ± 0.1 | Wild-type |

*¹threshold for sensor is 1.25 nA.
*²threshold for sensor is 0.42 nA

Table 2 shows the results of the analysis of BRAF mutations in cfNAs isolated and purified from melanoma patients, and also in unprocessed melanoma patient serum. As a control, serum from a healthy donor (HD) was processed and analyzed in the same way.

The sequences of the mutant BRAF allele, and of the oligonucleotides used as probes and clamps are shown below. DNA sequences are shown 5′ to 3′, and PNA sequences are shown N to C terminus. Underlined portions denote point mutations.

Probe for BRAF mutant
PNA Cys-Gly-GAT TTC TCT GTA GCT A (799 T > A)
Clamp for BRAF wild type
PNA GAT TTC ACT GTA G
Target for BRAF mutant
DNA TAG CTA CAG AGA AAT C
Target for BRAF wild type
DNA TAG CTA CAG TGA AAT C

For each sample analyzed using the electrochemical clamp assay, a mean signal of −1.0±0.3 nA (plus three standard deviations) measured in the healthy donor's sample was used as a cutoff value for determining the presence or absence of the BRAF mutation. A sample with a current level higher than the cutoff value is positive for the BRAF mutation, whereas a sample with a current level lower than the cutoff value is negative for the BRAF mutation. A previously-validated clamp PCR method was used to confirm the presence or absence of the BRAF mutation. In this clamp PCR method, when ΔCt-1≧2, the sample is positive for the BRAF mutation, and when ΔCt-1<0, the sample is negative for the BRAF mutation. The results of the electrochemical clamp assay and clamp PCR are comparable. The electrochemical clamp assay was also used to detect BRAF mutations in unprocessed melanoma patient serum. As demonstrated in all three assays, three (3) of the seven (7) melanoma patient samples were positive for BRAF mutation. The signal changes observed in electrochemical assay for the undiluted serum is lower than in the processed samples, which is expected due to much lower levels in the purified sample. Unlike the clamp PCR method, the electrochemical clamp assay is able to detect mutated BRAF in unprocessed serum.

TABLE 2
Analysis of BRAF mutations in samples from melanoma patients

| | Purified nucleic acids | | | | Serum | |
|---|---|---|---|---|---|---|
| | Electrochemical | | | | Electrochemical | |
| | clamp chip*¹ | | Clamp PCR | | clamp chip*² | |
| Samples | Δi (nA) | Assessment | ΔCt-1 | Assessment | Δi(nA) | Assessment |
| 1 | 5.3 ± 0.5 | BRAF mutated | 4.9 | BRAF mutated | 1.7 ± 0.3 | BRAF mutated |
| 2 | 7.9 ± 0.8 | BRAF mutated | 5.3 | BRAF mutated | 1.2 ± 0.6 | BRAF mutated |
| 3 | 0.8 ± 0.3 | Wild-type | −1.4 | Wild-type | −0.5 ± 0.1 | Wild-type |
| 4 | 0.6 ± 0.8 | Wild-type | −3.1 | Wild-type | 0.3 ± 0.4 | Wild-type |
| 5 | 1.2 ± 0.7 | Wild-type | −2.1 | Wild-type | −1.4 ± 0.2 | Wild-type |
| 6 | −1.1 ± 1.0 | Wild-type | −1 | Wild-type | −0.1 ± 0.2 | Wild-type |
| 7 | 21.9 ± 1.5 | BRAF mutated | 2 | BRAF mutated | 2.1 ± 0.3 | BRAF mutated |
| HD | 0.05 ± 0.6 | Wild-type | −1.2 | Wild-type | 0.3 ± 0.1 | Wild-type |

*¹threshold for sensor is 2.46 nA.
*²threshold for sensor is 0.69 nA.

III. Distinguishing Between DNA or RNA cfNAs

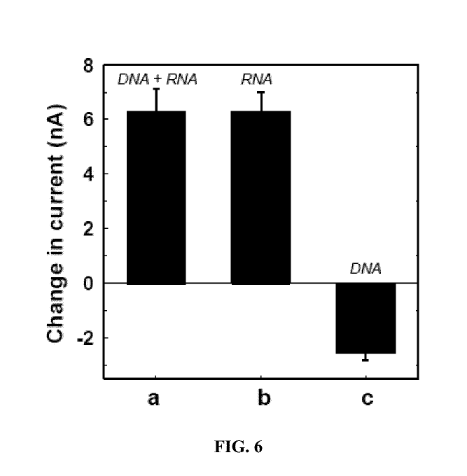

To identify whether the electrochemical clamp assay detects genomic DNA or transcribed RNA analytes the assay was challenged with cfNAs, cfNAs digested with DNase I, and cfNAs digested with RNase A. As illustrated in FIG. 6, the change for total cfNAs and cfRNAs are similar, whereas no signal change was observed for cfDNA. It is therefore concluded that the analytes detected are predominantly cfRNAs.

### IV. Materials and Methods

Materials

HAuCl₄, potassium ferricyanide (K₃[Fe(CN)₆), and hexaamine ruthenium (III) chloride (Ru(NH₃)₆Cl₃) were obtained from Sigma-Aldrich. ACS-grade acetone, isopropyl alcohol (IPA), and perchloric acid were obtained from EMD; hydrochloric acid was purchased from VWR.

Phosphate-buffered saline (PBS, pH 7.4, 1^{X}) was obtained from Invitrogen. All of the PNA probes and PNA clamps were obtained from PNA Bio, USA. PCR primers, synthetic DNA targets, and DNA clamps were obtained from ACGT, Canada. The A549 lung cancer cell line (catalog number CCL-185) and cultured medium (F-12K medium, catalog number 30-2004 supplemented with 10% (v/v) FBS and 5% CO₂ atmosphere) were obtained from ATCC, Canada. Lung cancer and melanoma patient serums were obtained from Bioreclamation Inc., USA.

Chip Fabrication

Six-inch silicon wafers were passivated using a thick layer of thermally grown silicon dioxide. A layer of about 25 nm Ti was deposited. A gold layer of about 350 nm was deposited on the chip using electron-beam-assisted gold evaporation. The gold film was patterned using standard photolithography and a lift-off process. A Ti layer of about 5 μm was deposited. A layer of insulating Si₃N₄ of about 500 nm was deposited using chemical vapor deposition; apertures of about were imprinted on the electrodes using standard photolithography, and bond pads of about 0.4 mm×2 mm were exposed using standard photolithography.

Fabrication of NMEs

Chips were cleaned by sonication in acetone for about 5 min, rinsed with isopropyl alcohol and DI water, and dried using a flow of nitrogen. Electrodeposition was performed at room temperature; 5 pm apertures on the fabricated electrodes were used as the working electrode and were contacted using the exposed bond pads. Au sensors were generated using a deposition solution containing a solution of about 50 mM HAuCl₄ and about 0.5 M HCl using DC potential amperometry at about 0 mV for about 100 s. After washing with DI water and drying, the Au sensors were coated with Pd to form nanostructures by replating in a solution of about 5 mM H₂PdCl₄ and about 0.5 M HClO₄ at about −250 mV for about 10 s. The control of sensor surface area has been characterized extensively and in this study, the average surface area was 4.75±0.3×10⁻⁴ cm² as determined by electrochemical Pd oxide stripping.

Clamp Chip Protocol

A 2 μM probe solution in water was prepared from a 20% acetonitrile solution containing about 100 pM PNA probe. Probe solutions were then heated to about 65° C. for about 5 min and chilled on ice for about 5 min before deposition. About 50 μL of the probe solution was dropped onto the chips and incubated for overnight in a dark humidity chamber at room temperature for immobilization of probe. The deposition used lead to a surface coverage of about 2×10¹³ molecules/cm². The chip was washed for about 10 min with PBS at about 60° C. followed by washing for about 10 min at room temperature. After initial electrochemical scanning the chips were then treated with different targets at about 60° C. Optimal hybridization time was determined to be about 15 min. After washing for about 10 min with PBS at about 55° C., followed by washing for about 10 min at room temperature of the chip, a final electrochemical scan was performed.

cfNAs Isolation from Exosomes

MW9 mutant BRAF 1799A melanoma exosomes and U373v3 glioblastoma exosomes (wild type BRAF and wild type KRAS control) were obtained from the laboratory of Prof. Janusz Rak's (Montreal Children's Hospital Research Institute, McGill University). MW9 and U373v3 exosomes were isolated by ultracentrifugation method and RNA was extracted by Trizol (Invitrogen). A549 exosomal RNA (mutant KRAS 134A) and exosomal RNA from patient serums was extracted, using Norgen biotek kit catalog number 51000. Isolated RNA had a A260/A280 ratio>2, indicating a high level of purity.

cDNA Synthesis and Clamp PCR

A volume of 2 purified cfNA (30-754 ng) was used for cDNA synthesis, in 20 μL reaction, with random hexamer primers and Superscript III reverse transcriptase, Invitrogen kit. A volume of about 2 μL cDNA was used in 50 μL not-competitive clamp PCR reaction with about 2 μM final concentration of gene specific primers, or in a 201.1 of real-time clamp PCR reaction, Panagene kit.

Mutant BRAF and Mutant KRAS Clamp Optimization

To validate that 60° C. was an appropriate specific temperature for the sensor assay, clamp PNA was tested in a qualitative PCR assay. The PCR program was as follows: template denaturing at about 95° C. for about 3 minutes followed by about 35 cycles of template denaturing at about 95° C. for about 30 seconds, primer annealing and DNA chain extension at about 60° C. for about one minute. The PCR products were visualized using agarose gel electrophoresis. PCR primers for BRAF (95 bp PCR product): Forward primer: FPBRAF3 (5′-CCT-CAC-AGT-AAA-AAT-AGG-TGA-TTT-TGG-3′), Reverse primer: RPBRAF3 (5′-CAC-AAA-ATG-GAT-CCA-GAC-AAC-TGT-TC-3′). PCR primers for KRAS (80 bp PCR product): Forward primer: FPKRAS (5′-GCC-TGC-TGA-AAA-TGA-CTG-AAT-ATA-3′), Reverse primer: RPKRAS (5′-TTA-GCT-GTA-TCG-TCA-AGG-CAC-TC-3′).

Real-Time Competitive Clamp PCR

Mutant BRAF and mutant KRAS real-time competitive clamp PCR were performed using a Panagene kit (mutant BRAF product number PNAC-2001 and mutant KRAS product number PNAC-1002). The real time clamp PCR was performed on ABI 7500 thermocycler and the SYBR Green reading was set at about 72° C. The PCR program was: template denaturing at about 94° C. for about 5 min followed by about 40 cycles of template denaturing at about 94° C. for about 30 sec, PNA clamp at about 70° C. for about 20 sec, primer annealing at about 63° C. for about 30 sec and DNA chain extension at about 72° C. for about 30 sec.

KRAS and BRAF Detection in Whole Serum

For mutation detection in whole serum we co-deposited 6-mercaptohexanol (MCH) with the probe to minimize nonspecific binding. For KRAS mutation detection, we used a universal probe for KRAS point mutations that was a combination of all of the possible mutant probes. An aqueous solution containing about 2 μM of PNA probes was heated to about 65° C. for about 5 min and, after annealing, about 18 μM of MCH was mixed with this probe solution. The solution was dropped onto chip and left overnight, followed by washing as described above. Serum samples were prepared by adding about 12.5 μL of lysis buffer (1×PBS containing about 10% NP40 and about 10% Triton X100), about 1 μL of 10 μM clamps for wild-type, and about 3 μL of RNAase inhibitor (Ambion, Am 2694) to about 50 μL of patients' serum. After initial electrochemical scanning, the above serum sample was dropped onto chip and incubated at about 60° C. for about 15 min. After washing, a final electrochemical scan was performed.

Electrochemical Analysis and Scanning Electron Microscopy (SEM)

All electrochemical experiments were carried out using a Bioanalytical Systems Epsilon potentiostat with a three-electrode system featuring a Ag/AgCl reference electrode and a platinum wire auxiliary electrode. Electrochemical signals were measured in a 0.1×PBS containing about 10 μM [Ru(NH₃)₆]Cl₃, and about 4 mM K3[Fe(CN)₆]. Differential pulse voltammetry (DPV) signals were obtained with a potential step of about 5 mV, pulse amplitude of about 50 mV, pulse width of about 50 ms, and a pulse period of about 100 ms. Signal changes corresponding to specific target were calculated with background-subtracted currents: change in currents=(I_{after}−I_{before}) (where I_{after}=current after target binding, I_{before}=current before target binding). SEM images were obtained using an Aspex 3025 SEM.

Variations and modifications will occur to those of skill in the art after reviewing this disclosure. The disclosed features may be implemented, in any combination and subcombination (including multiple dependent combinations and subcombinations), with one or more other features described herein. The various features described or illustrated above, including any components thereof, may be combined or integrated in other systems. Moreover, certain features may be omitted or not implemented. All references cited are hereby incorporated by reference herein in their entireties and made part of this application.

Examples of changes, substitutions, and alterations are ascertainable by one skilled in the art and could be made without departing from the scope of the information disclosed herein. All references cited herein are incorporated by reference in their entirety and made part of this application.

1. A detection system for electrochemically detecting a variant of a target sequence in a sample, the target sequence being present as a plurality of variants within the sample, the system comprising:
an electrode comprising a first probe on its surface, said probe being capable of binding a first variant of the target sequence and;
a second probe capable of binding a second variant of the target sequence, wherein the second probe is added to the sample, thereby preventing binding of the second variant to the first probe.

2. The detection system of claim 1, wherein the electrode is a nanostructured microelectrode.

3. The detection system of claim 1, wherein the variant of the target sequence is a cell-free nucleic acid (cFNA).

4. The detection system of claim 1, wherein the cFNA is released from a tumor.

5. The detection system of claim 1, wherein the sample is unprocessed serum.

6. A method for electrochemical detection of a variant of a target sequence in a sample, the target sequence being present as a plurality of variants within the sample, the method comprising:
contacting an electrode comprising a first probe on its surface with the sample, said first probe being capable of binding a first variant of the target sequence,
adding a second probe to the sample, said second probe being capable of binding a second variant of the target sequence, thereby preventing binding of the second variant to the first probe; and
measuring an electrochemical signal generated by the binding of the first variant of the target sequence to the first probe, wherein the electrochemical signal is indicative of the presence of the first variant within the sample.

7. A method according to claim 6, wherein the variant of the target sequence is a cell-free nucleic acid (cFNA).

8. The method of claim 6, wherein the cFNA is released from a tumor.

9. The method of claim 7, wherein the sample is unprocessed serum.

10. A point-of care diagnostic device configured to perform the method of claim 6.

11. A kit comprising:
a biosensor comprising an electrode;
a first probe affixed to surface of the electrode, said first probe being capable of binding a first variant of a target sequence in a sample, said sample containing a plurality of variants of the target sequence;
a second probe, capable of binding a second variant of a target sequence in a sample containing a plurality of variants of the target sequence, thereby preventing binding of the second variant to the first probe.

12. A kit according to claim 11, wherein the electrode is a nanostructured microelectrode.

13. A kit according to claim 11, wherein the first probe is capable of binding to a first variant of a gene, and the second probe is capable of binding to a second variant of a gene.

14. A kit according to claim 11, wherein the gene is a cancer-related gene selected from the group consisting of the KRAS gene and the BRAF gene.

15. A method of detecting a variant of a cancer-related sequence mutation in a sample from a patient, the method comprising the step of;
contacting an electrode comprising a first probe on its surface with the sample, said first probe being capable of binding a first variant of the cancer-related sequence mutation,
adding at least a second probe to the sample, said second probe being capable of binding a second variant of the cancer-related sequence mutation, thereby preventing binding of the second variant to the first probe; and
measuring an electrochemical signal generated by the binding of the first variant of the cancer-related sequence mutation to the first probe, wherein the electrochemical signal is indicative of the presence of the first variant within the sample.
